(19) United States
(12) Patent Application Publication
(10) Pub. No.: US 2015/0112358 A1
(43) Pub. Date: Apr. 23, 2015

(54) UMBILICAL CORD DRESSING

(71) Applicant: Bellie Buttin Marketing, LLC, Cosmos, MN (US)

(72) Inventors: John Miller, Cosmos, MN (US); James Miller, Stanford, KY (US); Jeannine Hehir, Burnsville, MN (US)

(73) Assignee: Bellie Buttin Marketing, LLC, Cosmos, MN (US)

(21) Appl. No.: 14/100,436

(22) Filed: Dec. 9, 2013

Related U.S. Application Data
(63) Continuation-in-part of application No. 29/470,371, filed on Oct. 21, 2013, now Pat. No. D722691.

(51) Int. Cl. A61B 17/122

(52) U.S. Cl. CPC A61B 17/122
USPC 606/120

(57) ABSTRACT

The present disclosure is directed to a novel way of dressing the umbilical cord stump of a newborn. In one aspect of the invention, the umbilical dressing cap is washable and reusable, and made to fit securely onto an umbilical cord clip to provide a clean and dry environment for the cord stump. In another aspect of the invention, the dressing is configured to receive printing to help identify the newborn. In yet another aspect of the invention, the dressing cap advantageously provides support for a flaccid cord stump.

### CLAIM OF BENEFIT TO PRIOR APPLICATIONS

This application claims the benefit of U.S. Design application Ser. No. 29/470,371 filed Oct. 21, 2013; and such application is hereby fully incorporated by reference herein.

### FIELD

The present invention relates generally to a dressing for the stump remains of the severed umbilical cord of a newborn. More particularly, the present invention relates to a dressing cap configured to fit over an umbilical cord clip.

### BACKGROUND

The human umbilical cord develops from and contains remnants of the yolk sac and allantois. It typically forms by the fifth week of fetal development, replacing the yolk sac as the source of nutrients for the fetus. The cord is not directly connected to the mother's circulatory system, but instead joins the placenta, which transfers materials to and from the mother's blood without allowing direct mixing.

The umbilical cord enters the fetus via the abdomen, at the point which (after separation) will become the umbilicus. Upon birth, the newborn's umbilical cord can be clamped at different times; however delaying the clamping of the umbilical cord until one minute after birth improves outcomes as long as there is the ability to treat jaundice if it occurs. Clamping is followed by cutting of the cord, which is painless due to the absence of nerves. The cord is extremely tough, like thick sinew, and so cutting it requires a suitably sharp instrument. While umbilical severance may be delayed until after the cord has stopped pulsing (5-20 minutes after birth), there is ordinarily no significant loss of either venous or arterial blood while cutting the cord.

The length of umbilical left attached to the newborn varies by practice; in most hospital settings the length of cord left attached after clamping and cutting is minimal, about 1 inch. The clip is typically placed about 1 inch above the stomach of the newborn to compress the umbilical cord, and hold it closed. The cord is then severed, thereby freeing the newborn baby from the placenta. Any remaining cord above the clip is trimmed with a knife to be leveled with the top surface of the clip. This remaining umbilical cord then needs to be kept clean and dry to prevent infection. The umbilical stub remains for up to 10 days as it dries and then falls off of its own accord.

Typically the remainder of the cord is simply clamped and left alone. In some instances the cord is dyed blue with a solution designed to reduce the risk of infection and promote drying. Generally however, cord care is such that the cord or stump is to be kept uncovered, clean and dry to prevent infection and promote rapid drying.

There exists in people a natural aversion to looking at and touching the uncovered umbilical cord stump. Many parents have a natural aversion to touching the cord or even looking at it, causing them to avoid the uncovered cord stump on newborn's body. This aversion can interfere with the parents' and siblings' bonding with their newborn at the critical first few hours of the newborns' life.

Further, during the changing of soiled and wet diapers the uncovered flaccid cord stump can be difficult to keep out of the way, making it difficult to keep the cord stump clean and dry.

Therefore, there is an unfulfilled need for a reusable cap dressing for an umbilical cord stump that can tend to support the stump, that can assist in keeping the stump clean and dry, that can make the cord stump more attractive, and that may be made baby specific to help identify and track newborns in busy hospitals.

### SUMMARY

The present disclosure is directed to a reusable cap for an umbilical cord stump configured to be attached to a standard umbilical cord clip to assist in keeping the cord stump clean and dry. Another aspect of the disclosure is that the invention is configured to accept printing that would serve to help identify the newborn. Another aspect of the disclosure is that the stump cap is also configured to assist in elevating the cord stump in a more upright position and away from the baby's stomach, and thus away from exposure to soiled diapers and from streams and pools of urine that may suddenly form.

Further, the present disclosure is directed to an umbilical cord dressing that is designed to cover the cord with a visually attractive cap that will cause the parents find the umbilical cord stump area more attractive, and thus less likely to interfere with the bonding process.

This summary is not intended to limit the scope of the invention, or describe each embodiment, implementation, feature or advantage of the invention.

### BRIEF DESCRIPTION OF THE DRAWINGS

FIG. 1 is a perspective view of an embodiment of the invention.

FIG. 2 is a side elevation view of an embodiment the invention.

FIG. 3 is an end elevation view of an embodiment the invention.

FIG. 4 is a bottom plan view of an embodiment of the invention.

FIG. 5 is a top plan view of an embodiment of the invention.

FIG. 6 is a perspective view of an embodiment of the invention in use.

FIG. 7 is a perspective view of an umbilical cord clip.

FIG. 8 is a perspective view of the clip clipped onto a cord stump.

FIG. 9 is a side elevation view of the dressing cap in use.

### DETAILED DESCRIPTION

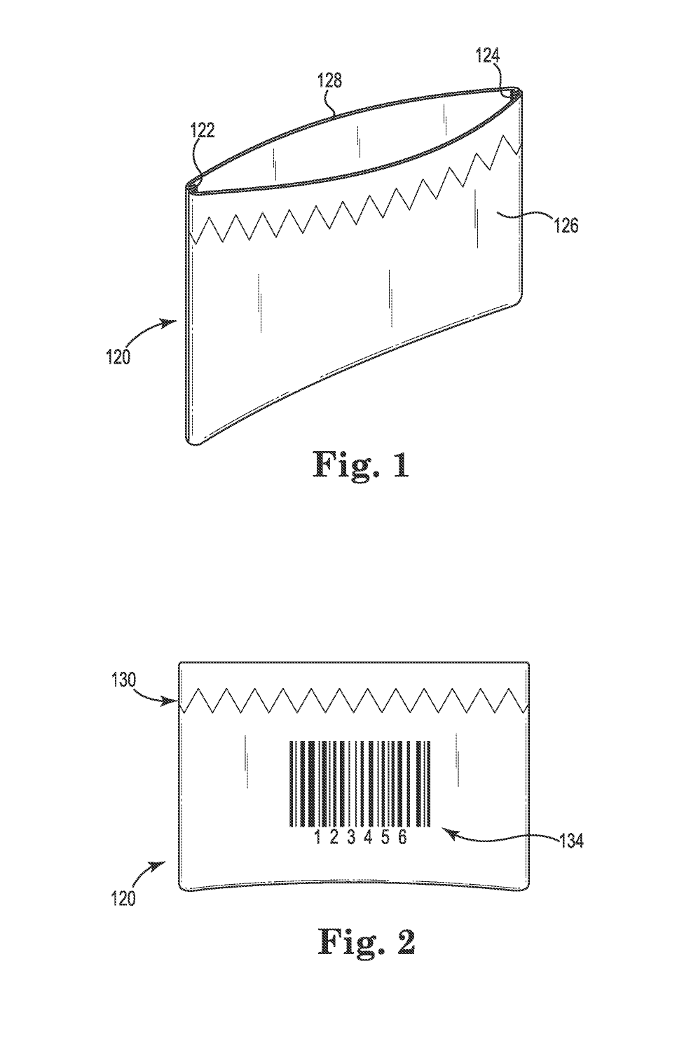

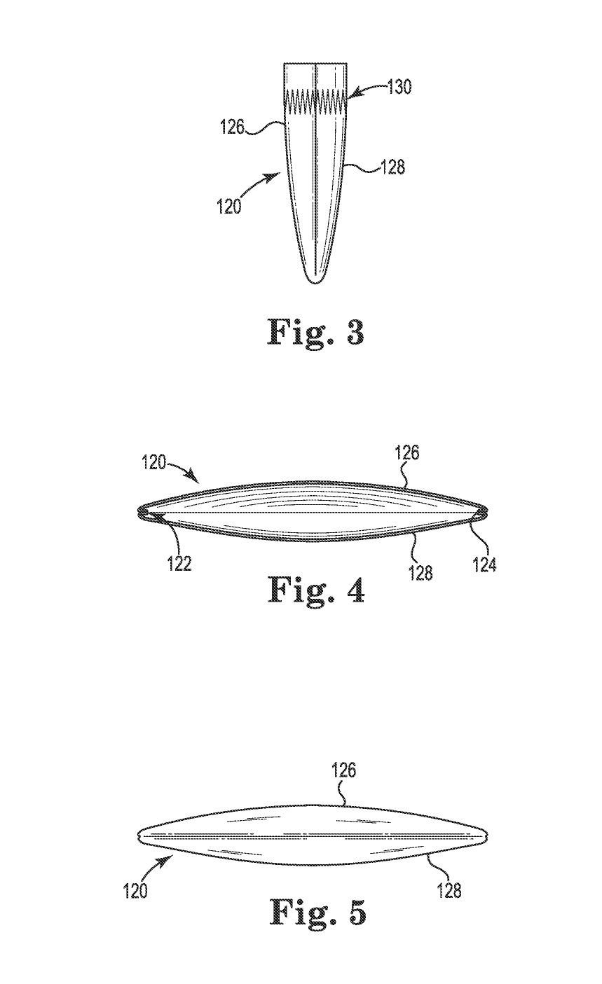

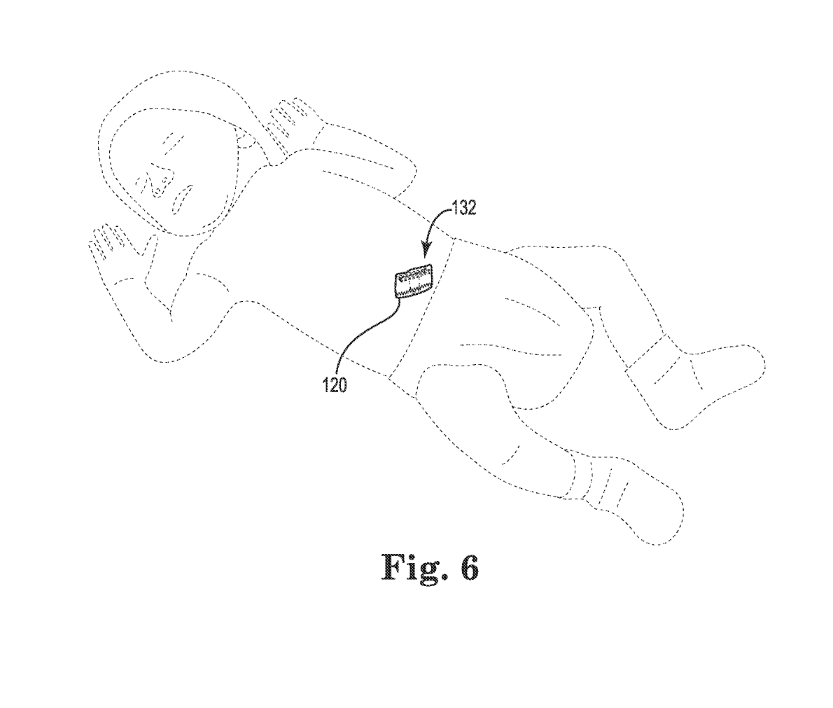

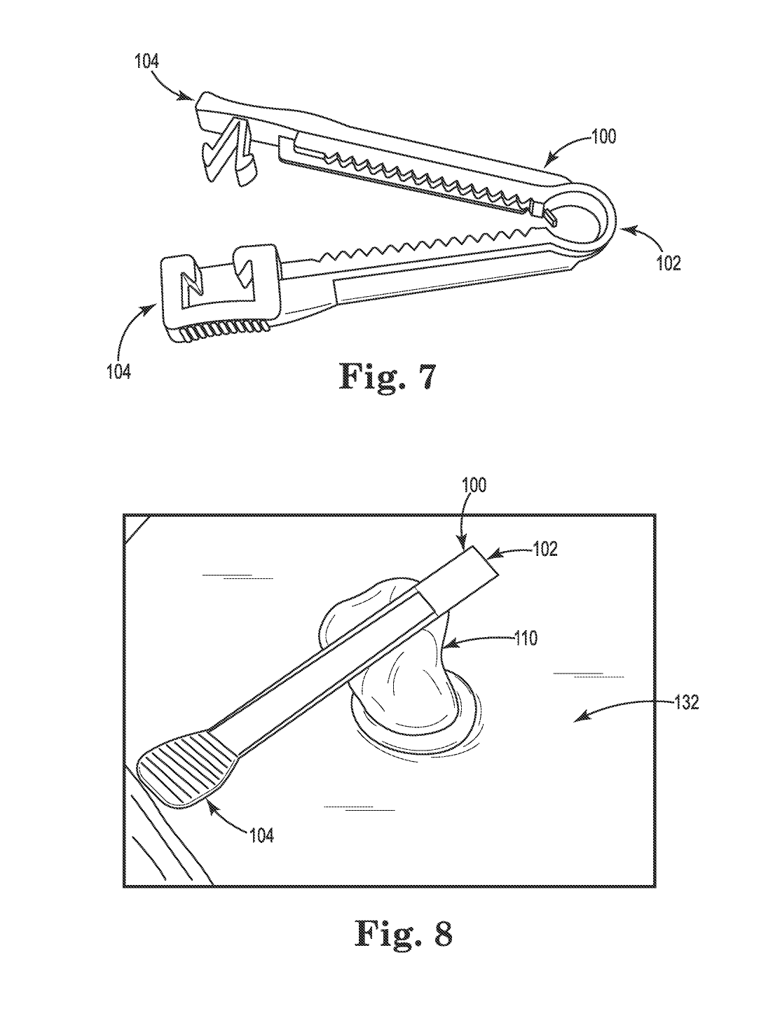

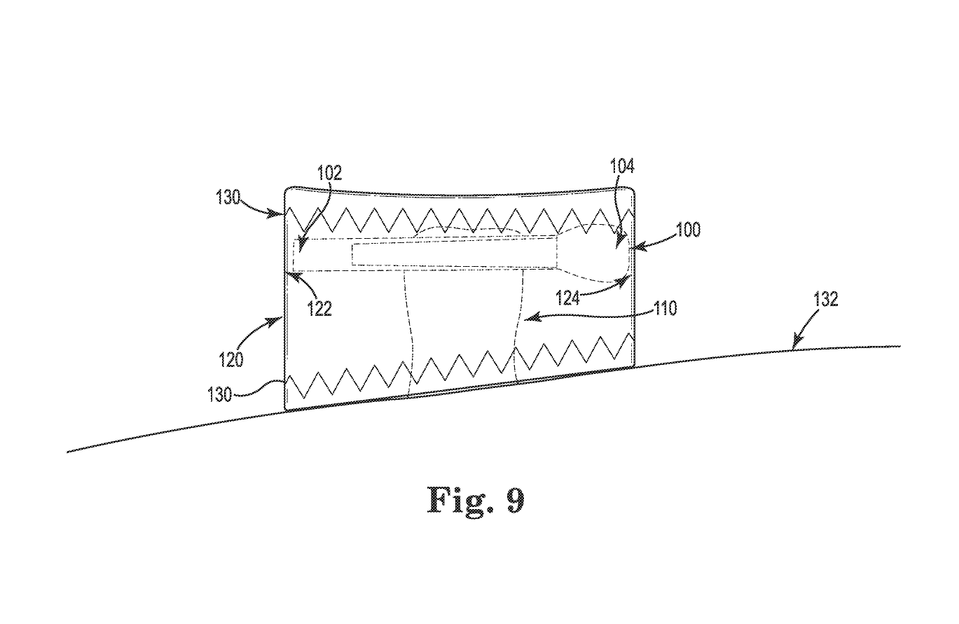

Referring to FIGS. 1-9, dressing cap 120 is configured to fit securely over clip 100, such that clip ends 102 and 104 frictionally engage the inside portions 122 and 124 of dressing cap 120. Clip 100 is attached to and is used to compresses umbilical cord stump 110.

Dressing cap 120 is preferably shaped similarly to a that of a garrison cap and is preferably constructed of a washable fabric such as cotton or a polyester material. Dressing cap 120 is configured to fit snuggly over clip 100 by stretching dressing cap 120 over clip 100 from end 122 to end 124 and pulling sides 126 and 128 downward toward the newborn's stomach 132.

The construction of the dressing cap 120 is such that it can accept printing, such as bar code 134. The newborn's name, address or other identifying information may also be applied. It is contemplated that such information can then be used to identify the newborn, or be used to cross-check the information on dressing cap 120 with the newborn's wrist or ankle band information to insure proper identification of the newborn.

The use of elastic 130 in the construction of dressing cap 120 is preferred to assist dressing cap 120 to obtain a snug fit over clip 100, such that clip ends 102 and 104 frictionally engage dressing cap 120, particularly at inside portions 122 and 124. It is preferred that the length of sides 126 and 128 be such that they extend to the baby's stomach 132, when dressing cap 120 is applied. This will assist dressing cap 120 in providing support to cord stump 110 and assist in maintaining cord stump 110 in a position away from stomach 132.

While the invention has been described in connection with what is presently considered to be the most practical and preferred embodiments, it will be apparent to those of ordinary skill in the art that the invention is not to be limited to the disclosed embodiments. It will be readily apparent to those of ordinary skill in the art that many modifications and equivalent arrangements can be made thereof without departing from the spirit and scope of the present disclosure, such scope to be accorded the broadest interpretation of the appended claims so as to encompass all equivalent structures and products. For example, the dimensions and proportions indicated in the figures may be altered without departing from the scope of the invention.

For purposes of interpreting the claims for the present invention, it is expressly intended that the provisions of Section 112, sixth paragraph of 35 U.S.C. are not to be invoked unless the specific terms “means for” or “step for” are recited in a claim.

What is claimed is:

1. A dressing for an umbilical cord stump comprising:
a cap shaped dressing having a top portion, an exterior portion and an interior portion;
the interior portion having a first interior end and a second interior end; and,
wherein the first and second interior ends of the cap are configured to frictionally engage a clip used to compress an umbilical cord stump of a newborn baby.

2. The dressing of claim 1 wherein the cap further comprises side portions configured to extend from the top portion to the stomach of the newborn baby.

3. The dressing of claim 2 wherein the cap is semi-rigid and wherein the engagement of the side portions of the cap with the newborn baby's stomach urges support for the umbilical cord stump.

4. The dressing of claim 1 wherein the cap further comprises markings on the exterior portion of the cap that can be used to identify the newborn baby associated with the cap.

5. The dressing of claim 1 wherein the cap is made of cotton or polyester.

6. A kit for dressing the umbilical cord stump of a newborn, comprising:
a cap shaped dressing having a top portion, an exterior portion and an interior portion,
a clip configured to lockably engage the stump remains of a severed umbilical cord of a newborn baby and having a first and second end when engaged, and;
wherein the cap is configured to fit over, and frictionally engage the first and second ends of, the clip.

7. A method of using a dressing for an umbilical cord stump of a newborn, comprising:
attaching an umbilical cord clip to the umbilical cord of a newborn baby approximately 1 inch above the stomach of the newborn;
cutting the umbilical cord above the clip; and,
placing a cap shaped dressing over the clip such that the cap frictionally engages the clip.

8. A dressing for an umbilical cord stump comprising:
a cap shaped dressing having an exterior surface and an interior diameter; and, wherein
the interior diameter of the cap is configured to frictionally engage a clip used to compress an umbilical cord stump of a newborn baby.

9. The dressing of claim 8 wherein the cap further comprises markings on the exterior surface of the cap that can be used to identify the newborn baby associated with the cap.
